(19) United States
(12) Patent Application Publication
(10) Pub. No.: US 2004/0059191 A1
(43) Pub. Date: Mar. 25, 2004

(54) Mechanical steering mechanism for borescopes, endoscopes, catheters, guide tubes, and working tools

(76) Inventors: Robert Krupa, Leominster, MA (US); William LaFlash, Northbridge, MA (US); Matthew Maher, Worcester, MA (US); Thomas Root, Beverly, MA (US); Ralph Tillinghast, Hardwick, NJ (US)

Correspondence Address: MIRICK O'CONNELL, DEMALLIE & LOUGEE, LLP, 1700 WEST PARK DRIVE, WESTBOROUGH, MA, 01581-3941, US

(21) Appl. No.: 10/462,951

(22) Filed: Jun. 17, 2003

Related U.S. Application Data
(60) Provisional application No. 60/389,168, filed on Jun. 17, 2002.

(51) Int. Cl. A61B 1/00

(52) U.S. Cl. 600/146

(57) ABSTRACT

A mechanism for articulating the distal end of an elongated tool through movement of drums, in which wires coupled to the drums connect the mechanism to the distal end of the tool that is being articulated. The mechanism comprises a joystick coupled to a rotatable shaft that is coupled to one of the drums. When the joystick is moved in one direction, the shaft is rotated, thereby rotating the drum and causing articulation in one plane. There is an arc arm rotatable by movement of the joystick. The arc arm rotates about an axis that is transverse to the shaft rotational axis. Rotation of the arc arm is accomplished by movement of the joystick in a plane that is transverse to the plane in which the joystick is moved to cause rotation of the first rotatable shaft. A gear system translates rotation of the arc arm to rotation of the second drum, to cause articulation in an articulation plane that is perpendicular to the articulation plane caused by rotation of the first drum. The result is an intuitive mechanical connection between the joystick and the articulating head that is simple to use and provides direct mechanical feedback from the articulating head to the user's hand.

### CROSS REFERENCE TO RELATED APPLICATION

This application claims priority of Provisional application serial No. 60/389,168, filed on Jun. 17, 2002.

### FIELD OF THE INVENTION

The invention relates to mechanical devices that are operated to accomplish remote articulation of the articulating end of a tool.

### BACKGROUND OF THE INVENTION

Endoscopic devices are commonly used in medical applications and non-medical applications. Typically, medical endoscopes are used to deliver and protect visual and/or medical devices in a patient (e.g., a human patient, an animal patient). Examples of medical endoscope devices include endoscopes (e.g., laparoscope, colonoscope, sigmoidoscope, bronchoscope) and catheters (e.g., optical, visual, ultrasonic). Often, non-medical endoscopes are used to inspect relatively difficult to view places, such as mining drill holes, the interior of an aircraft engine, or pipes. Examples of non-medical endoscopes include borescopes and fiberscopes. Both the medical and non-medical endoscopes vary in length and diameter depending on their application. An articulating or bending section is found at the distal end of some endoscopes. This bending section is controlled at the proximal end by a mechanism. This mechanism allows the operator of the scope to direct the distal end into the desired areas in which the endoscope has been placed (e.g., lungs, car engine). Typically this mechanism is found in three versions: one-way, two-way or four-way articulation. This represents the directions that the distal end can be moved. A fourth variation, utilized only with a joystick mechanism, is all-way articulation. FIG. 1 demonstrates these configurations of the distal end.

The distal end is typically articulated by pulling on wires that are held inside the insertion tube portion of the endoscope. These wires are connected to swing arms or drums that are moved or rotated by knobs, wheels, triggers, or levers. FIG. 2 shows a typical endoscope 500 with four-way articulation. This endoscope consists of two knobs 280, 290 (or, alternately, two levers or wheels) that are turned individually or simultaneously to move distal end 310 into the desired position.

The movement of the direction of the distal tip of a remote imaging device, commonly referred to as articulation, is most often accomplished by pushing and/or pulling wires attached between the distal tip of the endoscope and a gear system in the proximal handle. Gears (e.g., capstans, rack and pinion, cams) within the handle are moved by the operator using levers or wheels connected to the gears. In four-way articulation, the endoscope deflection is in two independent, perpendicular planes (e.g., left-right and up-down). In order to view a particular area that requires travel in both planes of movement, the operator must actuate two levers or knobs, usually in succession. This is cumbersome and not an intuitive process. Alternatively, an electronic joystick is employed that converts the more intuitive joystick movement into an electrical signal that can be processed and converted into electrical signals that drive a motor (for one-way and two-way articulation in a single plane) or two motors (for four-way and all-way articulation). The drawback with this means of articulation is the endoscope handle is typically connected (via an umbilical or tether) to an external power supply and processing electronics for the joystick and motors. This limits the portability of the device and the operator's access to remote locations. Alternatively, the motors, electronics, and power supply (e.g., batteries) are contained within the handle, making the device heavy, large, and difficult and tiring to use. Additionally, the operator lacks the “tactile feel” or feedback inherent in a mechanically actuated device that is often necessary to sense the device's advancement or resistance.

### SUMMARY OF THE INVENTION

The invention relates to the mechanism used to articulate the distal end of an elongated tool. The term “tool” as used herein includes, e.g.: remote imaging devices such as endoscopes, catheters, borescopes, and fiberscopes; optical measuring devices such as transmission, absorbance, reflectance, fluorescence and Raman devices; and ultrasonic imaging devices such as cardiac catheters, transesophageal ultrasonic imaging systems, and remote non-imaging devices such as insertion tubes, guide tubes, catheters, tools and devices placed down the working channel of catheters, endoscopes, borescopes and fiberscopes, laparoscopy tools and devices, tools and devices manipulated through glove box enclosures, and also, in general, any elongated device that is operated remotely, in which distal end articulation is necessary.

The inventive mechanism moves the articulating end in all four directions within the nominal sphere of the distal end. The invention uses a joystick lever approach to articulate the distal end tip. The mechanism is a two-axis, mechanically actuated device that allows the user to rotate two drums, cams, or gears (all termed herein “drums”). The particular type of drum used is based upon the diameter, length, and size of the tool. The drums are moved individually or simultaneously in either direction (e.g. clockwise or counter clockwise) by applying manual pressure to a joystick lever in the direction of desired articulation. This rotation pulls and/or pushes the wires connected to the distal end of the tool, causing the distal end to articulate to a desired position. This articulated movement permits the user to direct the view and/or placement of an instrument on the surface of an imaginary sphere. This invention relies upon the mechanical force generated at the joystick by the operator's hand, rather than relying on an electronic joystick that converts the joystick movement to an electrical signal, proportional to the joystick movement, that is used to drive an electronic motor or motors. This mechanical joystick, therefore, provides an intuitive direction with which the distal tip location can be interpolated based upon the joystick location. Additionally, the operator maintains a tactile sense or “feel” for the advancement through and the placement of the distal tip's environment.

This manual joystick mechanism is unique in that the joystick position is representative of the position of the distal tip of the tool, making operation of the tool much more intuitive and easier to use. In addition, this is the only mechanism that provides a nominally spherical surface of operation. This all-way articulation can be viewed as movement of the distal tip in an R-Theta (radius and angle) or spherical coordinate system. This is differentiated from typical four-way articulation, which is movement of the distal tip along two independent perpendicular planes (e.g., the XZ and YZ planes where the tool axis lies along the Z-axis). While both four-way and all-way articulation have similar end results (i.e., the distal tip can be moved to similar positions), only the all-way joystick mechanism accomplishes this in a simple, single step movement, whereas the four-way mechanism must make two independent movements to arrive at the same place in space.

### BRIEF DESCRIPTION OF THE DRAWINGS

FIGS. 1A through 1D are schematic diagrams illustrating the four typical articulation modes of a tool with a distal articulating head of the type in which the invention is useful;

FIG. 2 is a schematic diagram of a prior art tool with an articulating distal end, showing one manner in which the user accomplishes articulation;

FIG. 3 is a partial schematic diagram of one preferred embodiment of the mechanism of the invention for articulating the distal end of an elongated tool;

FIG. 4 shows an alternative arrangement to the mechanism of FIG. 3;

FIG. 5 is yet another alternative arrangement for the invention;

FIG. 6 is yet another alternative arrangement for the invention;

FIG. 7 is still another alternative arrangement for the invention;

FIGS. 8A and 8B are schematic views of one braking mechanism for the invention;

FIG. 9 is a schematic view of another braking mechanism for the invention;

FIG. 10 is a schematic view of yet another braking mechanism for the invention; and

FIG. 11 is a schematic view of yet another braking mechanism for the invention;

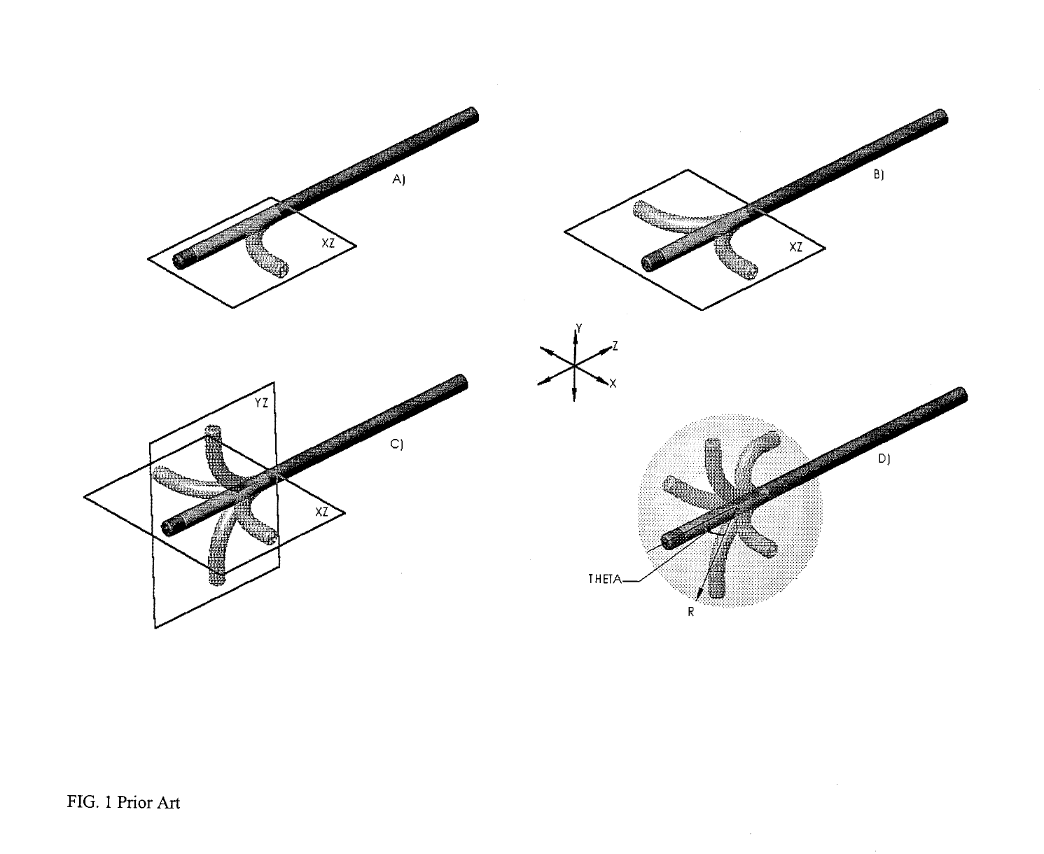

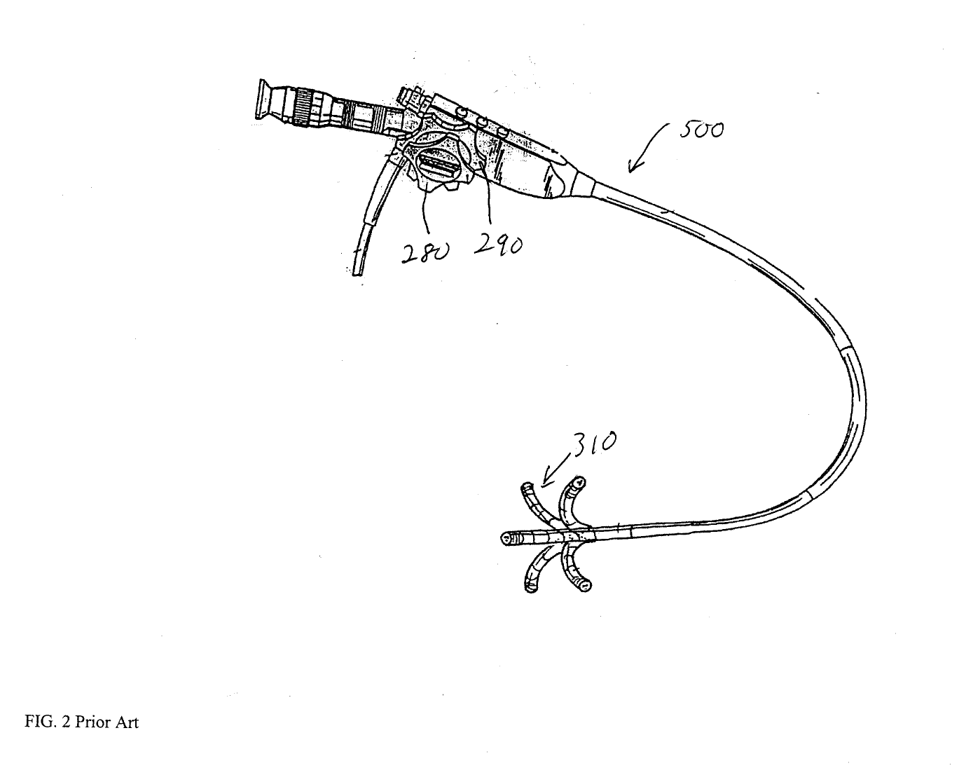

### DETAILED DESCRIPTION OF THE PREFERRED EMBODIMENTS

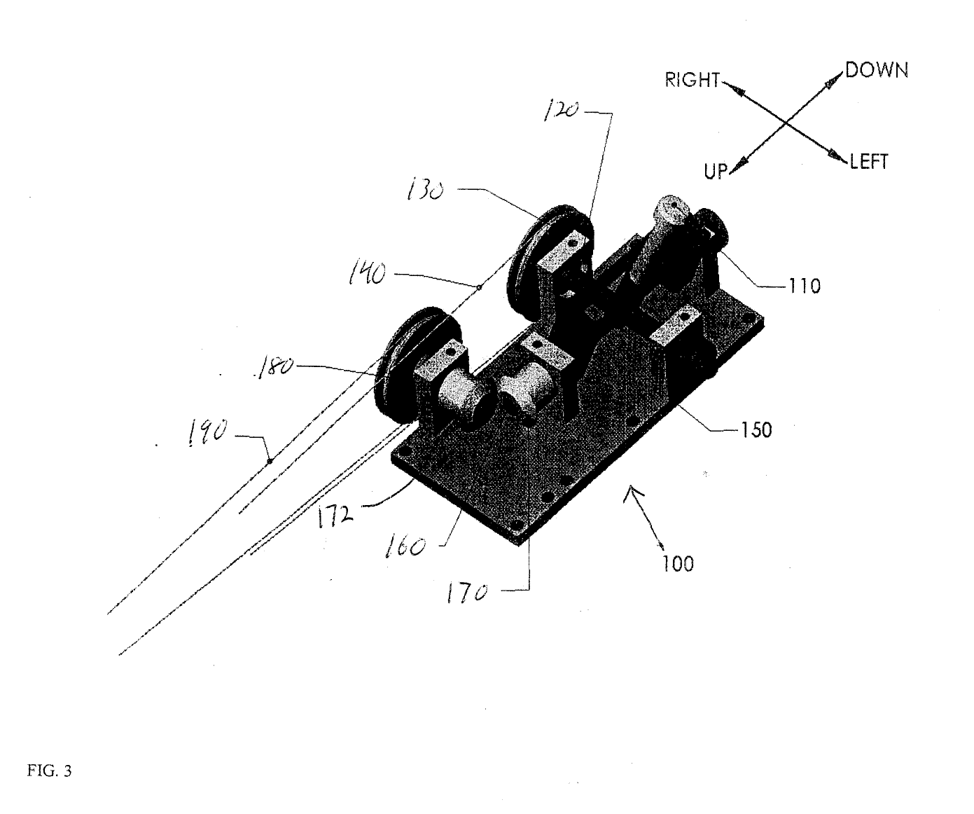

FIG. 3 shows a configuration of the articulation mechanism. The following is a breakdown of each part of the articulation mechanism.

Articulation Section:

The articulation section of the device can employ several different means of controlling the direction of the articulation. One method employs vertebrae that are capable of pivoting in a single plane (e.g., one-way and two-way articulation) or two nominally perpendicular planes (e.g., four-way and all-way articulation). An alternate method employs a softer and more flexible shaft material at the distal end of the device without the use of vertebrae. This method of articulation results in deflection of the distal tip of the device similar to that accomplished by articulation, but with less control over the direction or tracking (the ability to move the distal tip within a well-defined plane), and a lower angle of deflection. Articulation angles can be higher than 90 degrees when vertebrae are employed; without vertebrae, however, articulation is generally limited to less than 90 degrees of deflection.

Articulation Wire:

Articulation wires are typically attached to the distal tip of the tool, pass through an articulation section (e.g., vertebrae, spring guides, guide tubes), pass down the length of the shaft (sometimes through lumen in an extrusion, or through spring guides—flexible springs that will bend but not compress when the articulation wires are stressed), and ultimately to the proximal (handle) end where they are attached to a gear system. These wires typically range in diameter from about 0.008″ to 0.027″. These wires are typically made of steel or other metal alloys, but other materials such as Kevlar, Nitinol, nylon, rayon, and other polymer materials, as well as combinations of these materials can be used. The wires need to have minimal stretch to ensure that the articulation can be controlled. Typical elongation percentages for wire range from 1% to 4%.

Drums:

The articulation wires are connected to drums within the proximal end of the tool. These drums can range in diameter from about 0.5″ to 2″ depending on the application. The larger sizes are needed when large articulation angles are desired or long tool working lengths are used (longer lengths of tools require larger drums to take up the stretch in the articulation wire). The shape of the drum may also vary depending on the application. A cam shape may be desired to give the operator a mechanical advantage or to change the rate at which the distal end articulates during use. The drums are typically rotated 30 to 60 degrees in each direction, for a typical rotational range of 60 to 120 degrees. This rotation wraps the articulation wire around the circumference of the drum or cam, pulling on the distal articulated end of the device. This angle depends on the size of the drum and the application of the tool. Alternatively, the articulation wire may be pulled by a rack and pinion system, cam drive, planetary gear system, etc., determined by the force and travel required by the application.

Gear System:

A gear system is typically connected to each articulation drum. This can serve several purposes. First, a 90 degree rotation of one joystick axis may be desired so that both drums are directing the articulation wires along the tool's axis, in such a way as to have all four articulation wires parallel. Second, this gearing can be used to create a mechanical advantage such that less effort is needed when applying manual force to the joystick lever. Third, the gear ratio can be changed to allow a smaller diameter drum to be employed, but this increases the torque required to rotate that drum. A similar reduction can be accomplished using a planetary gear or rack and pinion mechanism.

Joystick Mechanism:

The joystick mechanism consists of a joystick lever which, when the user applies manual pressure, will either directly rotate one of the drums or rotate the arc arm which in turn will drive the gear system, thereby rotating the other drum. A universal swivel joint is located at the end of the joystick lever. This joint allows movement in one direction without effecting the other direction, thus allowing the drums to be rotated independently or simultaneously by the joystick lever, thereby providing all-way articulation rather than just four-way articulation along each plane. The length of the joystick lever can vary depending on the application of the tool. The movement of the joystick lever is limited by physical stops that are set by the assembler to ensure that the articulation will not damage the parts or other devices in contact with the articulating end. The joystick lever is typically moved (translated, displaced) 30 to 60 degrees in any one direction before hitting one of these stops. These stops can consist of limit screws, shaft collars, or other mechanical devices that will limit the joystick's, gears', and/or drums' ability to travel beyond a predetermined position.

FIG. 3 shows the preferred embodiment of the joystick device. Movement of joystick 110 in the up/down plane causes rotation of shaft 120 and drum 130. Up/Down articulation wires 140 are thereby pulled/pushed a distance proportional to the up/down movement of joystick 110. Movement of joystick 110 in the left/right plane causes rotation of arc arm 150, which translates this movement to shaft 160. Shaft 160 is attached to gear 170, which turns gear 172, which translates the rotation of shaft 160 by 90 degrees. Gear 172 further rotates drum 180, which pushes/pulls the left/right articulation wires 190. Movement of joystick 110 in the up/down plane thus causes tip articulation in only one plane (up/down), while joystick motion in the perpendicular right/left plane causes tip articulation in only the perpendicular right/left tip plane. Joystick motions that are not confined to a single plane cause motions of the tip in both planes. Since the joystick can be moved in two axes translationally, and in 360 degrees rotationally, the tip can be moved anywhere along its sphere. The tip motion is thus fully intuitive. Also, since the tip is moved fully mechanically, there is tactile feedback from the tip to the user's thumb operating the joystick, which helps to detect obstructions and the like.

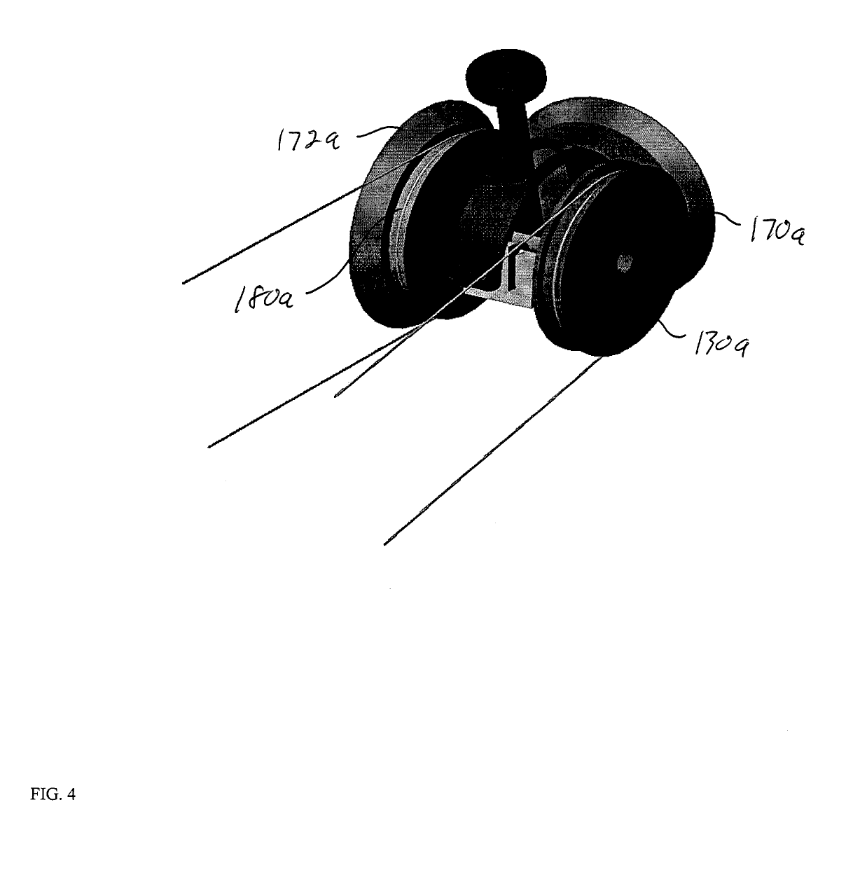

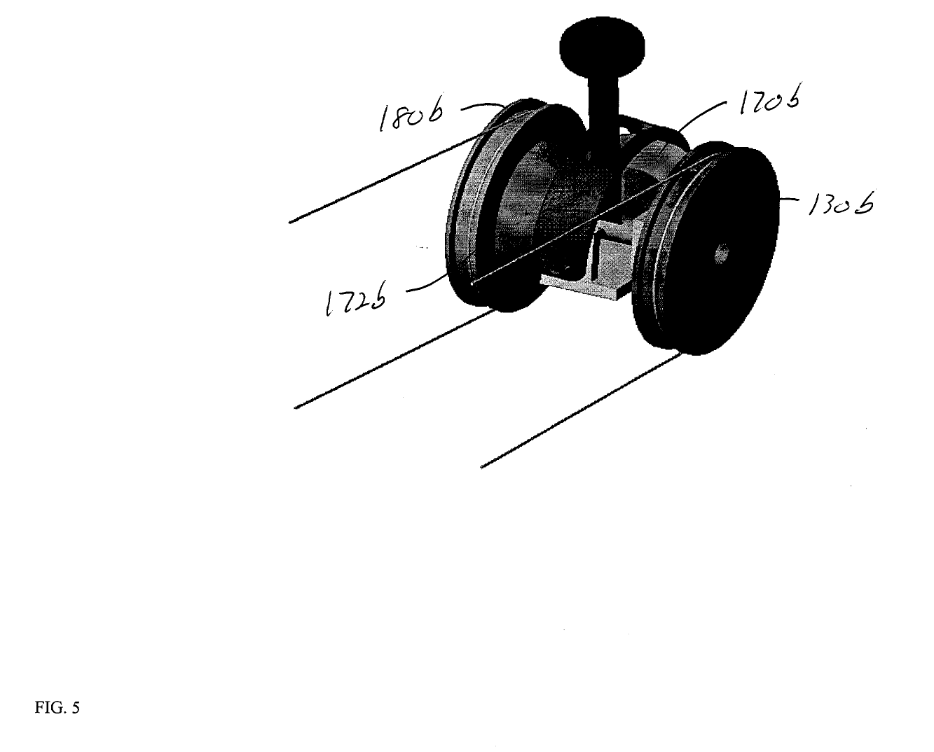

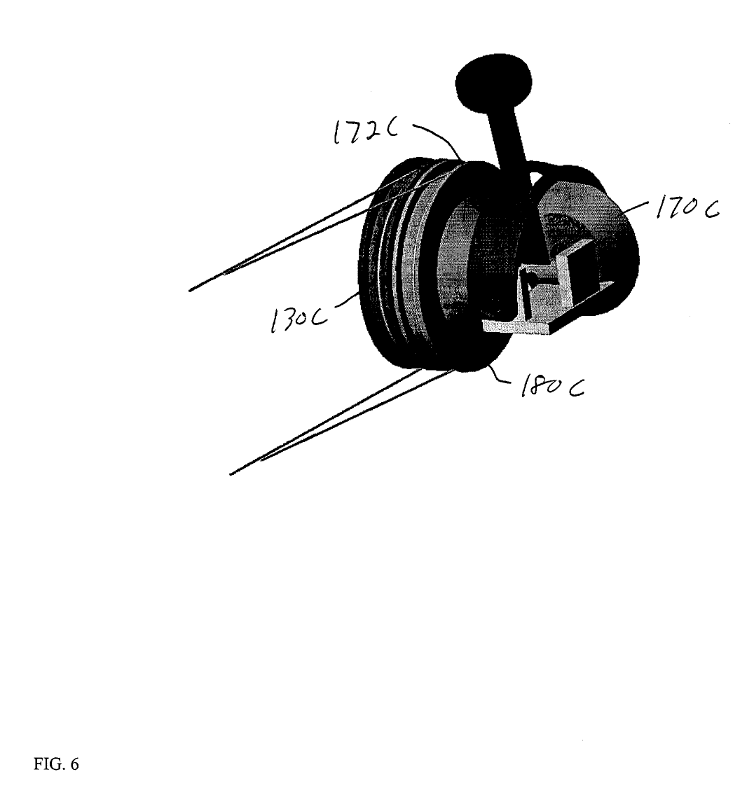

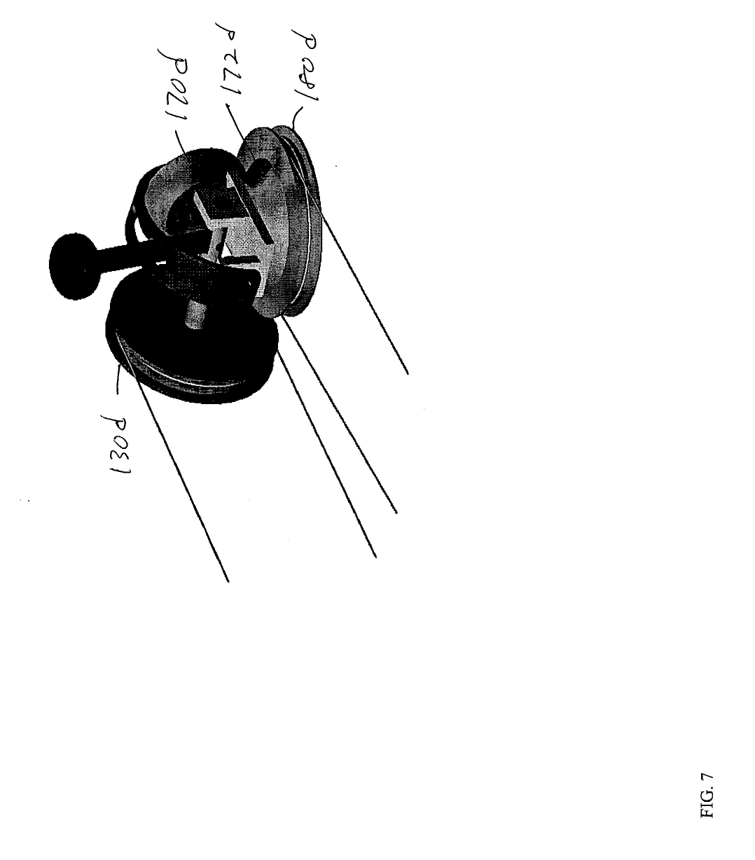

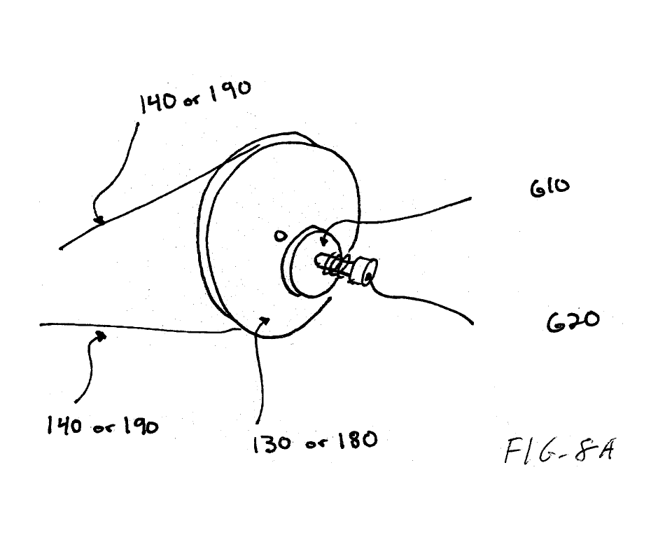

FIGS. 4 through 7 show other possible configurations for the inventive mechanism. FIG. 4 shows directly intermeshed gears 170a and 172a, with drum 180 coupled to gear 172a. FIG. 5 is very similar, but with intermeshed gears 170b and 172b inside of rather than outside of drums 130b and 180b. FIG. 6 shows a configuration in which the drums 130c and 180c are together. FIG. 7 shows a configuration in which drums 130d and 180d are in different planes. In this embodiment, the second gear 172d can be integral with drum 180d.

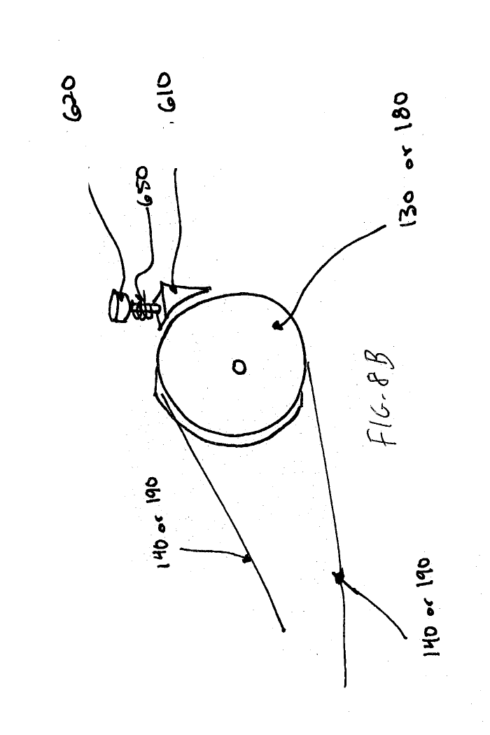

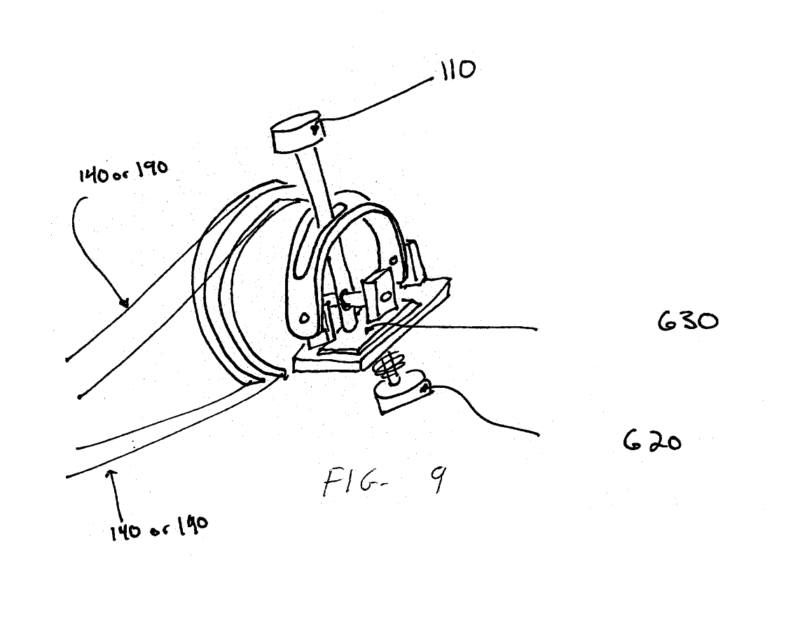

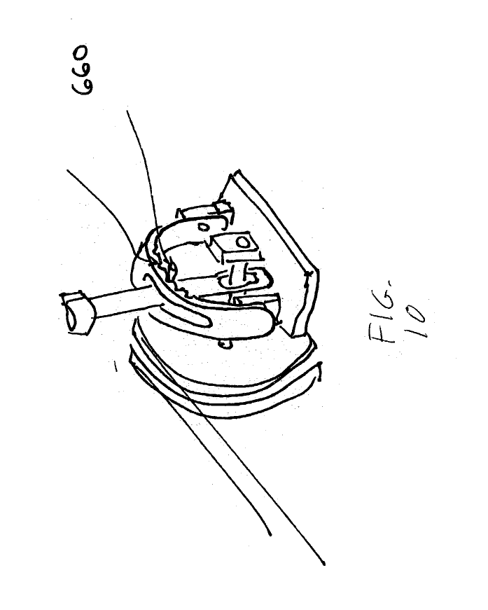

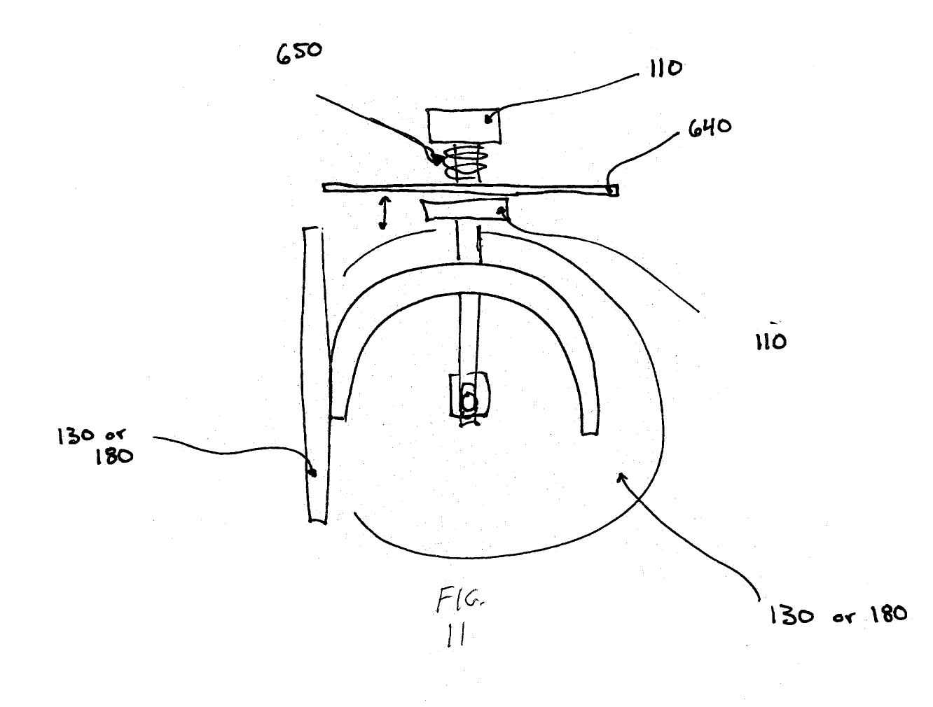

A braking mechanism is also included in the invention in which the articulation means is frozen or held in a particular position. This braking mechanism can take the form of: a friction brake (FIGS. 8A and 8B) in which a pad 610 is forced to contact the joystick 110, one or both of the drums 130 and 180, or one or both of the gears 170, 172; pushing the joystick down (FIG. 9), and latching this position, into a soft material 630 (e.g., a rubber pad) that holds the joystick position until the latch 620 is released; a ratchet mechanism 660, FIG. 10, on the gears and/or drums; or forcing the joystick up into a pad 640, FIG. 11 (e.g., a pad of soft rubber) via a spring 650, in such a way as to stop the joystick's movement until the joystick is pushed down (away from) this pad and allowed to move freely.

Other embodiments will occur to those skilled in the art and are within the following claims.

What is claimed is:

1. A mechanism for articulating the distal end of an elongated tool through movement of drums, with wires coupled to the drums and connecting the mechanism to the distal end of the tool, the mechanism comprising:
a rotatable shaft coupled to one drum;
a joystick coupled to the shaft, for rotating the shaft when the joystick is moved in a first plane;
an arc arm rotatable about an axis transverse to the shaft axis by movement of the joystick in a plane transverse to the first plane; and
a gear system for translating rotation of the arc arm to rotation of a second drum.

2. The articulation mechanism of claim 1 wherein the drums rotate about essentially parallel axes.

3. The articulation mechanism of claim 1 wherein the arc arm defines an opening through which the joystick passes.

4. The articulation mechanism of claim 3 wherein the joystick is coupled to the shaft through a universal swivel joint.

5. The articulation mechanism of claim 1 wherein the gear system comprises a first gear coupled to the arc arm and a second gear coupled to the first gear at an angle to the first gear.

6. The articulation mechanism of claim 1 wherein the tool is an endoscope.

7. An elongated tool with a mechanically-articulated articulating distal end, comprising:
at least two rotatable drums;
at least a pair of wires coupled to the drums, and also coupled to the tool's articulating distal end, for translating drum rotation into distal end articulation;
a mechanical joystick moveable translationally and through 360 degrees rotationally; and
a mechanism coupling the joystick to the drums, that mechanically translates motion of the joystick into rotation of the drums, wherein motion of the joystick in one plane causes rotation of only a first drum, and motion of the joystick in a perpendicular plane causes rotation of only a second drum, and movements of the joystick not wholly within these two planes causes rotation of both the first and second drums.
